(19) United States
(12) Patent Application Publication
(10) Pub. No.: US 2006/0204567 A1
(43) Pub. Date: Sep. 14, 2006

(54) Potato proteinase inhibitor II exhibits activity in elevating fasting plasma cholecystokinin concentrations

(76) Inventors: Jiang Hu, Ankeny, IA (US); Becky Edmondson, Des Moines, IA (US); Jennifer Radosevich, Ankeny, IA (US)

Correspondence Address: DAVIS, BROWN, KOEHN, SHORS & ROBERTS, P.C.;THE FINANCIAL CENTER, 666 WALNUT STREET, SUITE 2500, DES MOINES, IA, 50309-3993, US

(21) Appl. No.: 11/251,247

(22) Filed: Oct. 14, 2005

Related U.S. Application Data
(60) Provisional application No. 60/660,118, filed on Mar. 8, 2005.

(51) Int. Cl. A61K 9/48; A61K 36/81

(52) U.S. Cl. 424/451; 424/773

(57) ABSTRACT

A method of increasing fasting levels of cholecystokinin in a subject by the administration of potato proteinase inhibitor II is described. A method for extending satiety in a subject with elevated fasting cholecystokinin levels due to treatment is also described, along with a method of identifying subjects likely to respond to treatment.

This application claims priority to U.S. Provisional Application Ser. No. 60/660,118, filed Mar. 8, 2005.

### BACKGROUND OF THE INVENTION

The invention relates generally to plasma levels of cholecystokinin and, more specifically, to a method for raising fasting plasma cholecystokinin levels by the administration of effective amounts of potato proteinase inhibitor II (PI2).

Cholecystokinin (CCK), a well-studied gastrointestinal (GI) hormone, is involved in satiety and food intake regulation as well as blood glucose control in humans (Drucker, D. J. Enhancing incretin action for the treatment of type 2 diabetes. Diabetes Care. 2003 (26): 2929-2940; Liddle, R. A., Gertz, B. J., Kanayama, S., Beccaria, L., Gettys, T. W., Taylor, I. L., Rushakoff, R. J., Williams, V. C. and Coker, L. D. Regulation of pancreatic endocrine function by cholecystokinin: studies with MK-329, a nonpeptide cholecystokinin receptor antagonist. J Clin. Endocrin. & Metabol. 1990 (70): 1312-1318). Increased plasma CCK levels are able to delay gastric emptying, induce feeling of fullness and reduce food intake (Liddle, R. A., Morita, E. T., Conrad, C. K., Williams, J. A. Regulation of gastric emptying in humans by cholecystokinin. J Clin Invest 1986 (77): 992-996; Gutzwiller, J. P., Drewe, J., Ketterer, S., Hilderbrand, P., Beglinger, C. Interaction between CCK and a pre-load on reduction of food intake is mediated by CCK-A receptors in humans. Am J Physiol Regul Integr Comp Physiol 2000 (279): 189-195). Proteinase inhibitors of plant origin have been shown to elevate circulating CCK and in turn delay gastric emptying (Schwartz, J. G., Guan, D., Green, G. M., Phillips, W. T. Treatment with an oral proteinase inhibitor slows gastric emptying and acutely reduces glucose and insulin levels after a liquid meal in type II diabetic patients. Diabetes Care 1994 (17): 255-262). Oral administration of 1.5 g of potato proteinase inhibitor II (PI2) reportedly increased post-prandial CCK levels and reduced post-prandial hyperglycemia in type II diabetic patients (Schwartz, et al., 1994). Potato PI2 at the 1.5 g dose has also been shown to reduce energy intake in healthy lean subjects (Blundeii, J. E., Hill, A. J., Peikin, S. R., Ryan, C. A. Oral administration of proteinase inhibitor II from potatoes reduces energy intake in man. Physiol Behav 1990 (48): 241-246). Satiety-related GI hormones such as CCK have been suggested to have therapeutic value for obesity and diabetes. In diabetic patients with rapid gastric emptying, intervention to delay gastric emptying rate has been associated with improved control over post-prandial hyperglycemia and consequently hyperinsulinemia (Phillips, W. T., Schwartz, J. G., McMahan, C. A. Reduced postprandial blood glucose levels in recently diagnosed non-insulin-dependent diabetics secondary to pharmacologically induced delayed gastric emptying. Dig Dis Sci 1993 (38): 51-58; Phillips, W. T., Schwartz, J. G. Decelerating gastric emptying: therapeutic possibilities in type 2 diabetes. Diabet Med 1996 (13): S44-48). Unfortunately, such peptide hormones cannot be administered orally as they can be rapidly inactivated in the digestive tract.

Since we have shown that Pi2 induces endogenous CCK release and reduce post-prandial glucose levels, and can be orally administered, PI2 is an alternative treatment for weight loss and blood glucose control in obese and diabetic subjects.

### SUMMARY OF THE INVENTION

The invention consists of a method of increasing fasting levels of cholecystokinin (CCK) in a subject by administering to the subject an effective amount of potato proteinase inhibitor II (PI2). The PI2 is administered orally in an amount between about 1 and 1500 mg, preferably between about 1 and about 150 mg, and most preferably between about 5 and about 50 mg in human subjects. Ingestion of PI2 alone, without being accompanied by the ingestion of foods, beverages, or other nutritive compounds, was found to have the effect of increasing fasting levels of CCK. In a preferred embodiment, the PI2 is a powder that may be administered in either a capsule form or that can be added to foods or beverages.

Another aspect of the invention is a method of identifying subjects having a high increase in fasting plasma levels of CCK in response to the oral administration of PI2 prior to a meal by measuring plasma CCK levels in the subject prior to the administration of the PI2. People having a high level of fasting plasma CCK are more likely to benefit from ingestion of PI2.

A further aspect of the invention is a method for extending satiety following a meal by ingesting PI2 prior to the meal. The PI2 is administered orally in an amount between about 1 and 1500 mg, preferably between about 1 and about 150 mg, and most preferably between about 5 and about 50 mg in human subjects. Pre-prandial ingestion of PI2 alone, without being accompanied by the ingestion of foods, beverages, or other nutritive compounds, was found to have the effect of extending satiety following a meal, especially when pre-prandial levels of CCK were elevated due to prior ingestion of PI2. In a preferred embodiment, the PI2 is a powder that may be administered in either a capsule form or that can be added to foods or beverages. Ingestion of PI2 has been observed to extend satiety at least three hours following the meal.

### BRIEF DESCRIPTION OF THE FIGURES

FIG. 1 is a chart of the relationship of plasma CCK at the time of administration (T0) to the fasting CCK 60 minutes later (T-60) with three different treatments; each point represents a subject with a treatment; the regression lines show that CCK level at T0 is affected by the interaction of treatment and CCK level at T-60.

FIG. 2 is a chart of post-prandial plasma CCK response over 180 min among the three treatments; the concentration of CCK at the 15 mg dose was significantly different from the placebo.

FIG. 3 is a chart of post-prandial plasma CCK AUC at 0-90 min, 0-120 min and 0-180 min among the three treatments.

### DETAILED DESCRIPTION OF PREFERRED EMBODIMENTS

Potato proteinase inhibitor II (PI2) has been extracted from potatoes by a variety of methods. One such method is described in U.S. Pat. No. 6,767,566, which is incorporated herein by this reference. PI2 is available commercially from Kemin Consumer Care, L.C., Des Moines, Iowa, in tablets formulated to contain 15 mg PI2 per tablet and sold under the trademark Satise®.

### EXAMPLE I

Materials and Methods

Materials: Test articles in this study were supplied in size 0 gelatin capsules. Placebo capsules (Lot # KCC18-83-17JUNE04A and KCC10-194-22MAR04A) contained excipients including microcrystalline cellulose, magnesium stearate and silicon dioxide. PI2 capsules were comprised of potato protein extract containing 15 mg (Lot # KCC18-83-17JUNE04B and KCC10-194-22MAR04B) or 30 mg (Lot # KCC18-83-17JUNE04C and KCC10-194-22MAR04C) PI2 per capsule and excipients. The 390 kCal breakfast meal included 10 oz Tropicana Orange Juice and one serving of Good Start® Breakfast Meal (Aunt Jemima) containing bread, ham, egg, and cheese. The nutritional content of the meal is in Table 1.

Subjects: Fifty-five healthy female subjects of age 18-55 years and of BMI 19-29 were recruited. Forty-five subjects completed the study. Subjects were initially screened by blood and urine analysis of electrolytes, glucose, liver function tests, and general chemistries to ensure overall good health. Their body fat and lean mass were measured using bioelectrical impedance analysis (BIA). Their BMI, height, weight and medical history were also measured. Signed consents were obtained from subjects before the study began.

TABLE 1
Nutritional composition of the breakfast meal

| | |
|---|---|
| Nutrients per | |
| serving (g) | Total |

| | Breakfast | | | Energy |
|---|---|---|---|---|
| | meal | Juice | Weight (g) | (Kcal) |
| Protein | 12 | 2.5 | 14.5 | 58 (15%) |
| Fat | 9 | 0 | 9 | 81 (21%) |
| Carbohydrate | 30 | 32.5 | 62.5 | 250 (64%) |
| Total | 51 | 35 | 86 | 389 |

Procedures: This was a randomized, placebo-controlled, double-blind study. The Human Research Institutional Review Board (IRB) of Iowa State University approved the research protocol. Each subject was scheduled for a total of three visits separated by a 1-week washout period. Upon arrival after overnight fasting, 12 ml of blood was drawn from each subject. The subjects then consumed a treatment capsule that was randomly assigned as the placebo, 15 mg or 30 mg PI2. Sixty minutes later, a standardized 390 Kcal breakfast meal was served and subjects ate until satisfied but within 15 minutes after start of the meal. Any subject not consuming the entire meal was offered an equivalent calorie amount of alternative food to ensure the full 390 Kcal intake. No other food/drink was permitted during the visit except for 1 liter of bottled water. Blood samples were taken from each subject; the before meal sample was noted as time 0, and then 30, 60, 90, 120 and 180 minutes subsequently. Any adverse experiences that occurred during the study were recorded.

Measurements: The pre-meal and post-meal concentrations of CCK were evaluated up to 3 hours post-prandially. All blood samples were drawn into pre-coded and labeled Lavendar Vacutainer EDTA-tubes. The protease inhibitor aprotinin (Fisher Scientific, NJ) was added to a final concentration of 0.6 TIU/ml of blood. Each sample was gently mixed and immediately placed on ice. Within 1 h post-collection the sample was centrifuged at 3000 g for 15 minutes. The plasma was collected and stored at −80° C. for later,biomarker measurements. Plasma CCK was determined by radioimmunoassay using EURIA CCK kits (ALPCO Diagnostic, NH). Radioactivity was measured with a Packard Cobra II auto gamma counter (Perkin Elmer, Calif.). The CCK concentration expressed here represents the level of bioactive CCK-8 and equivalents in the plasma.

Data analysis: SAS software version 8.0 (SAS Institute Inc, Cary, N.C.) was used for all statistical analysis. The post-prandial plasma CCK data across time was analyzed using a cross-over analysis of variance containing the between-group factor of sequence (six different orders for three treatments consecutively experienced by each subject), the within-group factor of period (1, 2 and 3), and the within-group factor of treatment (placebo, 15 mg or 30 mg PI2) for the subjects. The areas under the curve (AUC) of the time courses for CCK were evaluated at post-prandial 90, 120 and 180 minutes using the model containing the between-group factor of sequence and the within-group factors of period and treatment. Additionally, the same analysis was used to evaluate the treatment effect on peak time (Tmax), peak concentration (Cmax), and pre-meal concentration at T0. The absence of a carry-over effect (i.e., the absence of influence of a prior treatment on a subsequent treatment) was assumed. The statistical significance was set at α=0.1. Results are displayed as least-square means (LSMEAN)±standard error of means (SEMs) unless noted otherwise.

### RESULTS

The average age for the forty-five subjects completing the study was 28.1±9.1 years, average weight was 66.6±11.9 kg, and BMI was 23.9±3.9 kg/m². The average lean body mass was 48.4±5.9 kg and the percentage of fat was 26.5±6.7 %. The average fasting CCK level was 0.45±0.87 pM. Overall, there was a significant main effect of time on post-prandial plasma CCK (p<0.01) in response to consumption of the 390 Kcal standard meal, showing that plasma CCK increased within 90 minutes after the meal and then decreased at 120 to 180 minutes.

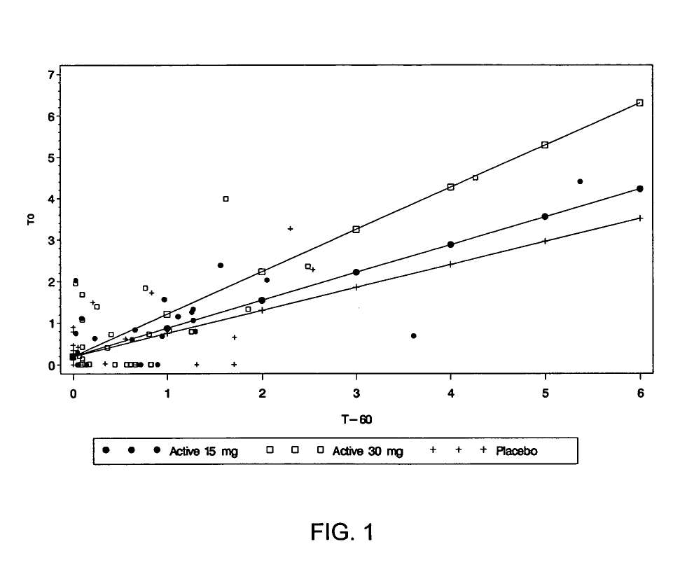

The effect of PI2 treatment on post-prandial concentrations of CCK at 0, 30, 60, 90, 120 and 180 minutes post-prandial and the changes from pre-meal baseline of integrated CCK area under the curve (AUC) were examined. A dose response of PI2 effect on the pre-meal CCK level at T0 was observed for placebo, 15 mg, and 30 mg PI2 doses with CCK levels of 0.45, 0.50 and 0.65 pM, respectively. The difference between 30 mg PI2 and placebo treatments reached significance with pair-wise contrasts (p=0.0825). This observation was affirmed by the interaction plot of treatments across CCK levels at T-60 (FIG. 1). The results indicate that ingestion of PI2 alone could raise the basal plasma CCK concentration to a higher level in 60 minutes. Moreover, the dose response of PI2 effect was increasingly well defined in subjects who exhibited higher baseline CCK at T-60, with relatively greater increases in CCK levels attained by pre-meal T0.

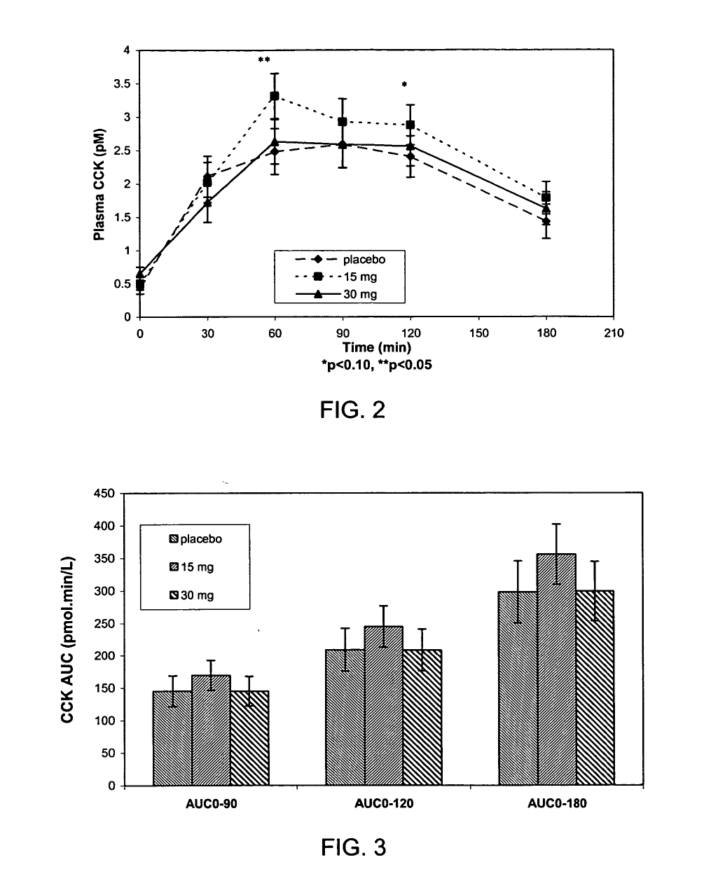

The post-prandial time-course of CCK is shown in FIG. 2. Post-prandial CCK levels were apparently higher with PI2 treatments than the placebo and this effect was more pronounced with the 15 mg dose. Individual contrast analyses revealed that 15 mg of PI2 induced significantly greater CCK elevation than the placebo at 60 and 120 minutes (p=0.0159 and p=0.0933, respectively). Fifteen mg of PI2 increased the mean CCK level 33.6% and 20.3%, respectively, relative to the placebo at these two time points. At 60 minutes, levels of CCK were 3.28±2.9, 2.74±2.0, and 2.40±1.7 pM (mean+SD) for the 15 mg, 30 mg, and placebo treatments, respectively. The change in CCK level from pre-meal T0 over the post-prandial period was also compared among the three treatments. The main treatment effect of PI2 was significant (p=0.0116) with the highest elevation of CCK found with 15 mg of PI2 (2.10 pM), followed by 30 mg (1.78 pM), and placebo (1.75 pM).

As shown in FIG. 3, oral administration of 15 mg of PI2 resulted in 16.9%, 17.2% and 19.4% increases in post-prandial CCK AUC at 0-90 minutes, 0-120 minutes and 0-180 minutes, respectively. When the average CCK level between T-60 and T0 was included as covariate in the model, post-prandial CCK AUC at 0-180 minutes was significantly higher with the 15 mg of PI2 treatment than placebo (p=0.0905). This supports a finding that the effect of PI2 on CCK release may be influenced by the average plasma CCK level observed at 1 hour before the meal. An interaction was found between the treatment effect and the fasting CCK level at T-60. According to the data, PI2 treatment resulted in increasingly higher AUC values relative to the placebo as fasting CCK levels increased. This was most pronounced with the 15 mg PI2 treatment. Thus, subjects responded better to PI2 treatments when they had a relatively higher fasting CCK level prior to treatment.

The average time at which CCK reached its peak level (Tmax) was 93.7±41.2 minutes, 91.0±38.6 minutes and 84.3±40.1 minutes (mean±SD) for the placebo, 15 and 30 mg treatments, respectively, indicating that PI2 might promote an earlier peak of meal-induced CCK in a dose-dependent manner. Peak concentrations of CCK (Cmax) were 3.5±2.1, 4.1±2.9 and 3.8±2.3 pM (mean±SD) for the placebo, 15 mg and 30 mg treatments, respectively.

### Discussion of Statistical Analysis

This section provides detailed analyses of the CCK values for the study described in this specification. The study utilized a three period cross over design with subjects randomly assigned to one of the six logical sequences in which three treatments (active 15 mg, active 30 mg and placebo) might occur. Analyses were conducted using a cross-over analysis of variance (ANOVA) containing the main effects of sequence, period and treatment. Typically subject values collected at a single point in time within a given period were evaluated using this model. In one instance, subject values across multiple time points within each period were analyzed. In this case, the main effect of time and the treatment by time interaction were added to the model. The absence of a carry-over effect was assumed for all cross-over ANOVA models given the short duration of effect that was expected of the active treatment and the use of adequate washout time intervals between periods. It is the treatment effect and any interaction involving treatment that form the focus of the analyses contained herein. Finally, an unstructured covariance matrix was assumed for models evaluating a single time point for each subject within each period. Compound symmetry was assumed when repeated measures for each subject within each period were analyzed.

1. Analysis of CCK Immediately Prior to the Test Meal

ANOVA results for CCK measured at zero minutes (just prior to the test meal), the model estimated means (Least Square Means or LSMeans) and all pair-wise contrasts between LSMeans with significance levels were calculated using an error term derived from the ANOVA table. The presence of a covariate (CCK_{T-60}) by treatment interaction (p=0.0070) and a significant contrast between the active 30mg and placebo groups (active 30 mg =0.65 pM, placebo=0.44 pM, difference=0.2088, p=0.0825) were found. From the borderline contrast, it is apparent that the mean CCK after receiving the treatment at −60 minutes has risen to a higher level in the active 30 mg group than in the placebo group. This observation is affirmed when the nature of the statistically significant covariate (CCK_{T-60}) by treatment interaction is plotted. FIG. 1 contains a scatter plot of the data being analyzed in each treatment across the values of the covariate along with the model predicted mean values (LSMeans) of each treatment across the values of the covariate. From this figure the nature of the covariate (CCKT_{T-60}) by treatment interaction can be understood. A dose response outcome (active 30 mg>active 15 mg>placebo) increasingly emerges over subjects who exhibit increasingly higher levels of baseline CCK (i.e., CCK_{T-60}).

2. Analysis of Post-Meal CCK Parameters

For the post-meal CCK parameters, the covariate by treatment (CCK_{T-60} by treatment) interaction for AUC₀₋₉₀, AU₀₋₁₂₀ and AUC₀₋₁₈₀ was significant. Significance levels for these interactions, respectively, are p=0.0069, p=0.0065 and p=0.0034. For AUC₀₋₉₀, AUC₀₋₁₂₀ and AUC₀₋₁₈₀ the underlying AUC values and the LSMeans for the three treatment groups across the values of the covariate (CCK_{T-60}). These figures indicate that the active 15 mg group increasingly exhibits over the covariate values a higher AUC than the placebo group. The same outcome pattern is observed for the active 30 mg group relative to placebo, but the effect is less pronounced. The covariate by treatment interaction observed when CCK_{T-60} serves as the covariate suggests that as the pre-treatment baseline level of CCK increases the presence of CCK (as measured by AUC over 90, 120 and 180 minutes) also increases in the active 15 mg group relative to placebo; and that as the pre-treatment baseline level of CCK increases the presence of CCK (measured by AUC over 90, 120 and 180 minutes) decreases less in the active 30 mg group relative to placebo.

3. Repeated Measures Analysis CCK Change Scores Relative to CCK_{T-60}, CCK_{T0} and CCK_{AVG} as Baselines

Three analyses that compare treatments on change scores computed by subtracting a baseline comprised of either CCK_{T-60}, CCK_{T0} or CCK_{AVG} from CCK measurements taken at 30, 60, 90, 120 and 180 minutes after the test meal (CCK Change Scores) were performed. The main effect of treatment was statistically significant for the CCK_{T0} Change Scores (active 15 mg=2.09pM, active 30 mg=1.71 pM and placebo=1.72pM, p=0.0116). Likewise for the CCK_{AVG} Change Scores the main effect of treatment was statistically significant (p=0.0280) with the highest mean CCK_{AVG} Change Score found in the active 15 mg group (2.0966 pM) followed by the active 30 mg group (1.7852 pM) and the placebo group (1.7474 pM).

### Discussion

Much evidence has indicated that PI2 ingestion induces satiety and reduces food intake in humans (Blundell, et al., 1990; Vasselli, J. R., Greenfield, D., Schwartz, L., Heymsfield, S. B. Consumption of a pre-meal drink containing protease inhibitor from potatoes decreases hunger and increases fullness in overweight subjects following a meal (Abstract). Presented at the North American Association for the Study of Obesity (NAASO) Annual Meeting 1999; Owyang, C. Discovery of a Cholecystokinin-Releasing Peptide: Biochemical Characterization and Physiological Implications. Ch. J Physiology 1999 (42): 113-120). One proposed mechanism is that PI2 inhibits the degradation of putative CCK releasing factors and subsequently enhances endogenous CCK release (Liddle, R. A. Regulation of cholecystokinin secretion in humans. Gastroenterology 2000 (35): 181-187; Owyang;, Herzig, K. H., Schön, I., Tatemoto, K., Ohe, Y., Li, Y., Fölsch, U. R., Owyang, C. Diazepam binding inhibitor is a potent cholecystokinin-releasing peptide in the intestine. Proc. Natl. Acad. Sci. USA 1996 (93): 7927-7932). Consistent with the hypothesis, PI2 at relatively high doses has been shown to increase post-prandial CCK level in humans. A 1.5 g dose of PI2 given with a liquid meal has reportedly increased circulating CCK level at 15 minutes post-prandial in type II diabetic subjects but did not affect the integrated post-prandial AUC (Schwartz, et al., 1994). Peikin et al. reported that the pre-meal ingestion of 1 g PI2 sustained a higher post-prandial CCK response than 1 g of lactose in healthy men given a 500 Kcal meal (Peikin, S. R., Springer, C. J., Dockray, G. J.,Calam, J. Oral administration of the proteinase inhibitor potato 2 stimulates release of CCK in man. Gastroenterology. Abstract, 1987 (92): A1570). Our study confirms that PI2 sustains a higher post-prandial CCK level for a longer period of time at doses much lower than previously snown.

The statistical analyses of the data contained herein support three main findings. First, that the impact of PI2 is most pronounced, both prior to and after a post-treatment meal, among subjects who are found to exhibit a non-zero value of CCK at baseline. The greater the baseline CCK value the greater the relative impact of active treatment on the subject. Second, the impact of PI2 just prior to ingestion of a one-hour post-treatment meal both depends on the baseline presence of CCK and the dosage level of PI2 (i.e., a dose response effect was observed prior to the post-treatment test meal). Third, PI2 impacts CCK post-meal values over a 180-minute post-meal measurement period. This post-meal effect is evident in the form of a covariate (CCK_{T-60}) by treatment interaction when the level of CCK is captured as AUC over part or all of the 180 minute post-meal evaluation period, and also evident when a baseline CCK value (CCK_{T0} or CCK_{AVG}) is subtracted from CCK at each post-meal time point (30, 60, 90, 120 and 180 minutes) and the change scores are analyzed.

Our results demonstrate that the fasting CCK is elevated 60 minutes after PI2 consumption in a dose-dependent manner in healthy women. It has been commonly believed for some time that cholecystokinin is released in the blood only as a function of the presence of digested lipids and/or protein in the duodenum (Burton-Freeman, B., Davis, P. A., Schneeman, B. O. Plasma cholecystokinin is associated with subjective measures of satiety in women. Am. J Clin. Nutr. 2002 (76): 659-657.; Burton-Freeman, B., Davis, P. A., Schneeman, B. O. Interaction of fat availability and sex on post-prandial satiety and cholecystokinin after mixed-food meals. Am. J Clin. Nutr. 2004 (80): 1207-1214). Previous analysis of human and animal experiments on regulation of CCK release suggested that proteinase inhibitors could stimulate CCK release in fasted rats, but that in humans there was a requirement of positive background nutrient stimulus of CCK release (Green G. M. Feedback inhibition of cholecystokinin secretion by bile acids and pancreatic proteases. In: Cholecystokinin, edited by Reeve, J. R. New York Academy of Sciences, 1994, p167-179. Liddle, R. A. Regulation of cholecystokinin secretion by intraluminal releasing factors. Am. J Physiol. 1995 (269): G319-327). This concept has been stated in several CCK physiology reviews (Liddle, R. A. Cholecystokinin cells. Annu. Rev. Physiol 1997 (59): 221-42. Moran, T. H. and Kinzig, K. P., Gastrointestinal satiety signal II. Cholecystokinin. Gastrointest liver Physiol 2004 (286): 183-188). However, our data instead show that PI2 alone can stimulate CCK release, indicating that the generally held notion that orally administered proteinase inhibitors need to be administered in conjunction with a meal to increase CCK levels is unexpectedly not valid. The reason for the observation that oral ingestion of PI2 results in increased pre-meal CCK levels in a dose responsive manner is unclear at present.

### SUMMARY

The results of this study showed that pre-meal ingestion of 15 to 30 mg of PI2 had an impact on fasting CCK concentrations before the meal, further enhancing post-prandial CCK in response to a meal in individuals with higher fasting CCK levels due to prior PI2 treatment. Increasing post-prandial CCK response has important implications in promoting satiety and reducing glycemic load, which in turn facilitate weight loss and weight control in humans. Therefore, PI2 may serve as an effective agent to promote weight loss and weight maintenance.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

We claim:

1. A method of increasing fasting plasma levels of cholecystokinin in a subject, comprising the step of orally administering to the subject potato proteinase inhibitor II (PI2).

2. A method as defined in claim 1, wherein the PI2 is in powder form for administration in capsule form or for addition to a food or beverage.

3. A method as defmed in claim 1, wherein the amount of PI2 is between about 1 and about 1500 mg.

4. A method as defined in claim 1, wherein the amount of PI2 is between about 1 and about 150 mg.

5. A method as defined in claim 1, wherein the amount of PI2 is between about 5 and about 50 mg.

6. A method of identifying subjects having a high increase in fasting plasma levels of cholecystokinin in response to the oral administration of PI2 prior to a meal, comprising measuring plasma cholecystokinin levels in the subject prior to the oral administration of PI2.

7. A method as defined in claim 6, wherein the measurement of plasma cholecystokinin is taken after the administration of PI2 but prior to a meal.

8. A method of identifying subjects most likely to benefit from the oral administration of PI2 to raise plasma cholecystokinin levels, comprising measuring the plasma cholecystokinin level of the subject prior to administration of PI2.

9. A method as defined in claim 8, wherein the measurement of plasma cholecystokinin is taken after the administration of PI2 but prior to a meal.

10. A method for extending satiety following a meal in a subject identified according to the method of either claims 6-9, comprising orally administering to the subject PI2 prior to the meal.

11. A method as defined in claim 10, wherein the PI2 is in capsule form.

12. A method as defined in claim 10, wherein the amount of PI2 is between about 1 and about 1500 mg.

13. A method as defined in claim 10, wherein the amount of PI2 is between about 1 and about 150 mg.

14. A method as defined in claim 10, wherein the amount of PI2 is between about 5 and about 50 mg.

15. A method as defined in claim 10, wherein satiety is increased at least 3 hours following the meal.
